(19) United States
(12) Patent Application Publication
(10) Pub. No.: US 2016/0346319 A1
(43) Pub. Date: Dec. 1, 2016

(54) NO/HE GAS MIXTURE WITH BACTERICIDAL ACTION

(71) Applicant: AIR LIQUIDE SANTE (INTERNATIONAL), Paris (FR)

(72) Inventors: Pierre DE VILLEMEUR, Louveciennes (FR); Laurent LECOURT, Boulogne (FR)

(21) Appl. No.: 15/114,915

(22) PCT Filed: Jan. 27, 2015
(86) PCT No.: PCT/FR2015/050182

§ 371 Date: Jul. 28, 2016

(30) Foreign Application Priority Data
Jan. 31, 2014 (FR) 1450770

(51) Int. Cl. A61K 33/00; A61K 9/00

(52) U.S. Cl. CPC A61K 33/00; A61K 9/007

(57) ABSTRACT

The invention relates to a gas composition containing nitrogen monoxide (NO) and helium (He), which is administered by inhalation in order to prevent or treat at least a bacterial infection affecting all or some of the respiratory tract of a patient, especially the bronchial tree and the lungs.

### CROSS REFERENCE TO RELATED APPLICATIONS

This application is a 371 of International PCT Application PCT/FR2015/050182 filed Jan. 27, 2015 which claims priority to French Patent Application No. 1450770 filed Jan. 31, 2014, the entire contents of which are incorporated herein by reference.

### BACKGROUND

The present invention relates to a gas composition based on nitric oxide (NO) and helium (He), used by inhalation to combat a bacterial infection of the respiratory airways of a patient, especially of the bronchial tree and of the lungs.

Nitric oxide (NO) is a gas customarily used by inhalation for treating hypoxemic respiratory distress linked to pulmonary vasoconstriction in humans, such as ARDS or persistent pulmonary hypertension of the newborn (PPHN). In this case, the doses administered to the patients rarely exceed 80 ppm by volume, the rest of the gas mixture inhaled being formed essentially of oxygen and nitrogen.

However, certain publications have shown an antibacterial effect of NO when it is used at a high dose, i.e. typically around at least 150 ppm by volume.

In this respect, mention may be made of the following documents:
H. Grasemann et al., Curr. Pharm. Des.; 2012; 18(5): 726-36; Nitric oxide and L-arginine deficiency in cystic fibrosis, et
C. Miller et al., Nitric Oxide. 2009 February; 20(1):16-23. Epub 2008 Aug. 26; Gaseous nitric oxide bactericidal activity retained during intermittent high-dose short duration exposure.

In these documents, the doses of inhaled NO leading to a bactericidal effect in the individuals tested are of the order of 160 to 200 ppm by volume for a continuous administration.

However, these high concentrations may lead to direct toxicities linked to the NO itself but also indirect toxicities linked to the formation of oxidized derivatives of NO, such as NO₂, by oxidation of the NO in contact with the oxygen present in the gas mixtures inhaled by the patients (i.e. which contain 21% by volume or more of oxygen) and, furthermore, an increase in the amount of methemoglobin which is undesirable.

Even though slightly better results, i.e. due to a lower formation of NO₂ and of methemoglobin, were obtained during a sequential administration of NO over a period of 30 minutes every 3 or 4 hours, it is important to be able to reduce the dose of inhaled NO administered in order to reduce the risk of toxicity for the patient.

The problem that is faced is hence to be able to use inhaled NO to combat a bacterial infection of the airways of a patient, especially of the bronchial tree and of the lungs, while reducing the associated risk of toxicity. In other words, it is desirable to be able to reduce the toxicity of the NO while retaining its bactericidal properties.

### SUMMARY

The solution is then a gas composition containing nitric oxide (NO) and helium (He) for use by inhalation for preventing or treating at least one bacterial infection affecting all or some of the respiratory airways of a patient.

Depending on the case, the gas composition of the invention may comprise one or more of the following technical features:
the respiratory airways comprise the bronchial tree and the lungs;
the content of NO is between 10 and 5000 ppm by volume (ppmv), preferably less than 3500 ppmv, more preferably less than 2500 ppmv, more preferably less than 2000 ppmv, more preferably less than 1500 ppmv;
the content of NO is between 10 and 1000 ppmv;
the patient is a human being, in particular an adult, a child or a newborn;
the NO/He gas mixture is diluted with an oxygen-containing gas in a ventilation circuit of a respirator or of a medical ventilator, for example with oxygen or an N₂/O₂ mixture such as air;
the gas composition additionally contains oxygen, preferably at least 21% by volume of oxygen;
the gas composition additionally contains nitrogen (N₂);
the gas composition Ia composition consists of helium, nitrogen and NO;
the NO/He gas mixture is packaged in a gas cylinder;
the NO/He gas mixture is used by being inhaled continuously or in a sequential manner;
the NO/He gas mixture is diluted with an oxygen-containing gas in a medical nebulizer;
the NO/He gas mixture is delivered continuously to the patient over a period of a few minutes to several tens of minutes;
alternatively, the NO/He gas mixture is delivered in a discontinuous or sequential manner to the patient, for a few minutes to several tens of minutes, over a time period of from one hour to several hours;
the NO/He gas mixture is administered in combination with an antibiotic treatment or product. Preferably, the antibiotic treatment or product combined with the NO/He gas mixture is selected so as to obtain a synergy of action with said NO/He gas mixture.

Generally, the invention also relates to a therapeutic treatment method, in which a gas composition according to the invention comprising a mixture of nitric oxide (NO) and helium (He) is administered by inhalation to a patient having a bacterial infection of the respiratory airways, especially of the bronchial tree and of the lungs, said gas mixture containing less than 5000 ppm by volume (ppmv) of nitric oxide (NO), and said patient being an adult, a child or a newborn.

Optionally, an antibiotic product or treatment is additionally administered to the patient, preferably in combination with the NO/He gas mixture so as to obtain a synergy of action in the elimination of all or some of the bacteria.

### BRIEF DESCRIPTION OF THE DRAWINGS

The present invention will now be better understood owing to the following description and the appended figures, among which:

FIG. 1 is a diagram of the respiratory airways of a human being,

FIG. 2 is an embodiment of equipment for administering an NO/He mixture according to the invention to a patient, and

FIG. 3 depicts the change in the pressure drop between the mouth and the alveolar zone of an individual for various gas flow rates.

### DESCRIPTION OF PREFERRED EMBODIMENTS

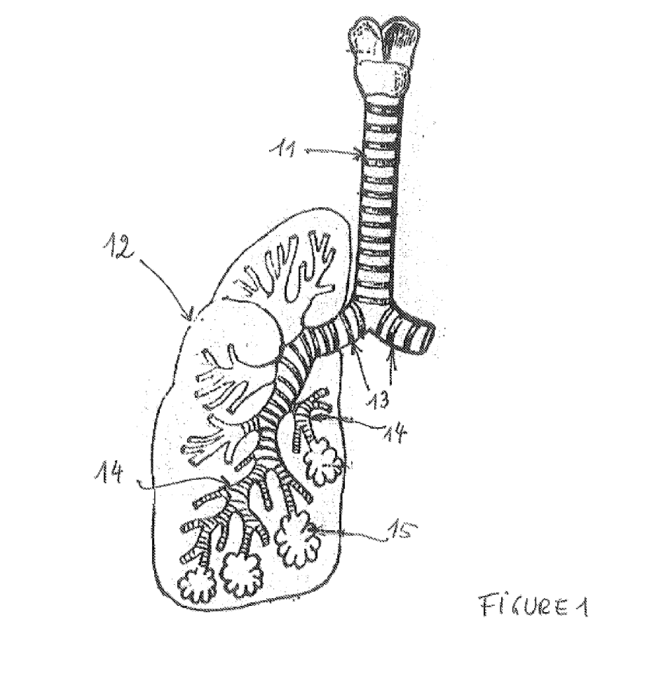

FIG. 1 represents a diagram of the respiratory airways of a human being revealing the trachea 11, the bronchi 13, the bronchioles 14 and the alveoli 15 of a lung 12. All these portions of airways are capable of being affected by a bacterial infection, in particular the bronchial tree and the lungs.

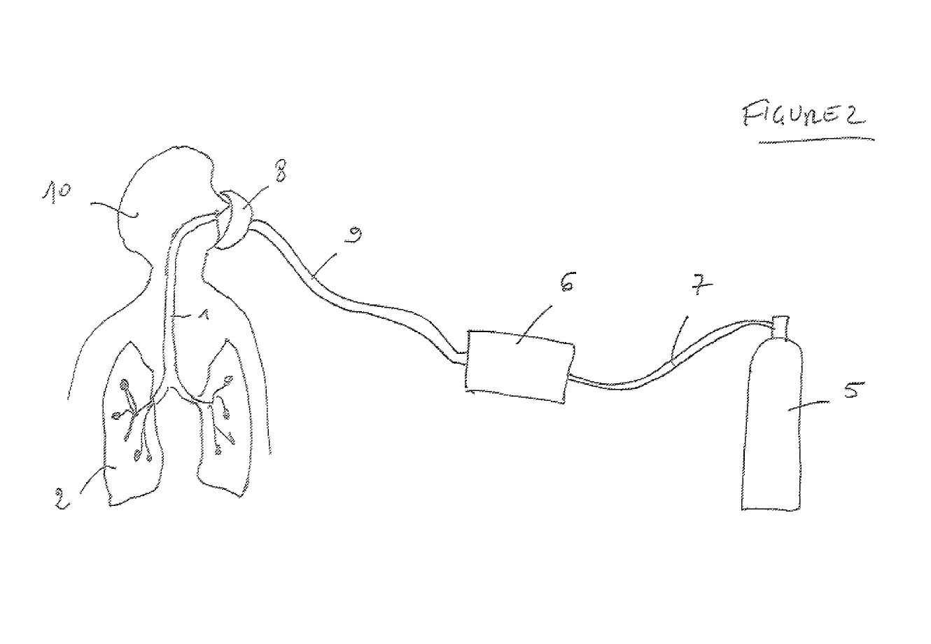

In order to combat such a bacterial infection, use is made according to the present invention of an NO/He mixture administered by inhalation, for example by means of the equipment from FIG. 2.

Seen therein is equipment for administering an NO/He mixture to a patient comprising a source 5 of a gas mixture formed from NO and helium containing for example from 10 to 4000 ppm by volume of NO, the rest being helium, typically from 10 to 1000 ppmv of NO.

This NO/He source 5, such as a gas cylinder, supplies, via a duct 7, a nebulizer 6 which is itself connected to a patient interface, such as a respiratory mask 8, by means of a flexible line 9 so as to be able to convey the gas to the airways of the patient 10, especially the bronchial tree 3 and the lungs 2.

More specifically, the administration of the NO/He mixture from the gas cylinder 5 to the patient 10 is carried out by diluting an initial NO/He mixture, for example containing from 10 to 4000 ppm by volume (ppmv) of NO and the rest being helium, typically from 10 to 1000 or 2000 ppmv, with oxygen-enriched air, i.e. that contains at least 21% oxygen, or more than 30% oxygen so as to obtain a given final concentration of NO, typically between 5 and 250 ppm of NO, which is administered to the patient by inhalation, preferably less than 150 ppm by volume of NO.

This dilution may be carried out at the pneumatic nebulizer 6 or by any other conventional means or device.

In fact, helium, owing to its physical properties, makes it possible to improve the flow regimes by favoring the laminar regime, during the inhalation thereof by an individual.

Owing to an NO/He gas mixture where helium replaces at least some, or even all, of the nitrogen generally used as carrier gas, the following are obtained: a better penetration of the NO at the bronchial level 13, 14, and consequently a reduction in the NO concentration needed to obtain an effective bacterial action, and a reduction in the concentration inhaled in order to obtain an identical effective dose at the alveolar level 15, given that the NO can then act up to the bronchial and alveolar levels.

### Examples

In order to demonstrate the advantage of an NO/He gas mixture according to the present invention in the treatment of a bacterial infection affecting all or some of the respiratory airways of an individual, e.g. a human patient, the following simulation tests were carried out, which are based on:
a calculation of the gas flow resistance in the respiratory airways of the individual, which may be linked to the respiratory function of the patient, i.e. the respiratory work thereof;
a calculation carried out on a model of bronchial obstruction of asthmatic type which is similar to that encountered in bacterial infections (i.e. bronchial inflammation and alveolar obstruction linked to the secretion of mucus or another factor).

### Gas Flow Resistance in the Respiratory System of an Individual

From a general point of view, the flow of a gas in a closed structure, such as the bronchial tree, loses a portion of its energy by viscous friction along the walls, i.e. linear pressure drop, and by any geometric irregularity that forces the fluid particles to change direction, i.e. singular pressure drop.

This is the case for example in the presence of a bifurcation, of a significant narrowing, typically a constriction, of an obstacle, etc.

This loss of energy, which is negligible in the case of a healthy individual, i.e. who is not infected by bacteria, may become critical in the case of a degradation of the bronchial structure, i.e. in an individual whose airways are infected by bacteria (leading to bronchoalveolar inflammation and obstruction) going as far as to give rise in the latter to an increase in the respiratory effort to be provided in order to inhale the same amount of gas.

Besides the morphological features, the physical properties of the gases, such as the density or the dynamic viscosity, influence these pressure drops: I. Katz et al., Property value estimation for inhaled therapeutic binary gas mixtures: He, Xe, N₂O, and N₂ with O₂. Med. Gas Res. 2011; 1:28.

The use of a gas having physical properties that reduce these pressure drops may then be beneficial to the patient.

Thus, from a quantitative point of view, by considering that the bronchial tree consists of a series of tubes and bifurcations, the pressure drop between one point of the respiratory system and the alveolar zone may be expressed by the following equation:

$$p - {p}_{alv} = ρ ( Σ H ) - ρ α \frac{{v}^{2}}{2}$$

where:
pₐₗᵥ is the alveolar pressure;
R is the density of the fluid (kg·m⁻²);
H is a pressure drop term;
ν is the velocity of the fluid (m·s⁻¹); and
α is a coefficient dependent on the type of flow.

The term H is the sum of the linear and singular pressure drops. It can be written in the form:

ΣH=Hₗᵢₙ+Hₛᵢₙ

where:
Hₗᵢₙ represents the linear pressure drops which, in case the tube, may be written:

$$\sum H = {H}_{lin} + {H}_{sin}$$

f is a friction coefficient dependent on the nature of the walls and the flow characteristics; L. Gouinaud et al., Inhalation pressure distributions for medical gas mixtures calculated in an infant airway morphology model. Comput Methods Biomech. Biomed. Engin. 2014 Apr. 4; 1-9;
L and D represent the geometric characteristics of the branch considered, i.e. length and diameter;
Hₛᵢₙ represents the singular pressure drops. In our case, it corresponds mainly to the presence of the bifurcations but may also be used to take into account particular constrictions (stenosis) or severe obstructions:

$${H}_{lin} = \sum f \frac{L}{D} \frac{{v}^{2}}{2}$$

the coefficient K is particularly difficult to predetermine since it depends very strongly on the geometric characteristics of the irregularity.

This coefficient K was calculated for a set of geometric configurations of industrial type, especially grid, tubing, etc.

A specific numerical simulation study made it possible to empirically determine this coefficient K for a set of geometries and flow conditions: I. Katz et al., The ventilation distribution of helium-oxygen mixtures and the role of inertial losses in the presence of heterogeneous airway obstructions; J. Biomech. 2011 April 44(6): 1137-43.

The results of these simulations made it possible to express the coefficient K, for a bronchial tree of a 9-month-old child, in the following form:

$${H}_{sin} = \sum K \frac{{v}^{2}}{2}$$

where: Re is the Reynolds number

$$K = \frac{B}{{Re}^{A}} + C {log ( Re )}^{2} + D log ( Re ) + E$$

μ being the dynamic viscosity of the fluid).

Table I below groups together the results obtained for the coefficients A, B, C, D and E in the case of the geometric characteristics of a 9-month-old child.

TABLE I

| Generation | D (m) | L (m) | N^{o} | A | B | C | D | E |
|---|---|---|---|---|---|---|---|---|
| Extrathoracic | 0.00449 | NA | 1 | 0.30902 | 0.19507 | 0.00992 | 20.08184 | 0.19314 |
| 0 | 0.006625 | 0.04521 | 1 | 0.36950 | 105.10240 | 1.12700 | 27.05540 | 9.43270 |
| 1 | 0.004812 | 0.01902 | 2 | 0.36130 | 204.42490 | 21.04450 | 13.44690 | 241.95410 |
| 2 | 0.003199 | 0.00759 | 4 | 0.35480 | 56.23990 | 2.04760 | 215.20890 | 28.38170 |
| 3 | 0.002213 | 0.00304 | 8 | 0.55750 | 464.34170 | 22.92850 | 24.66270 | 245.10450 |
| 4 | 0.001827 | 0.00507 | 16 | 4.70280 | 213.10074 | 2.57144 | 218.25327 | 32.99055 |
| 5 | 0.001374 | 0.00427 | 32 | 2.43100 | 23839.737 | 3.66111 | 223.96989 | 39.71066 |
| 6 | 0.001096 | 0.0036 | 64 | 5.43917 | 755.60625 | 0.94486 | 27.31618 | 14.16231 |
| 7 | 0.000881 | 0.00304 | 128 | 0.63982 | 25.32475 | 1.54187 | 29.44081 | 14.68968 |
| 8 | 0.00073 | 0.00256 | 256 | 2.65331 | 21768.064 | 22.70258 | 7.74494 | 1.70626 |
| 9 | 0.000605 | 0.00216 | 512 | 0.28046 | 272.55289 | 6.34181 | 242.07123 | 84.54093 |
| 10 | 0.000508 | 0.00184 | 1024 | 0.28046 | 272.55289 | 6.34181 | 242.07123 | 84.54093 |
| 11 | 0.000431 | 0.00156 | 2048 | 0.28046 | 272.55289 | 6.34181 | 242.07123 | 84.54093 |
| 12 | 0.000377 | 0.00132 | 4096 | 0.28046 | 272.55289 | 6.34181 | 242.07123 | 84.54093 |
| 13 | 0.000332 | 0.00108 | 8192 | 0.28046 | 272.55289 | 6.34181 | 242.07123 | 84.54093 |
| 14 | 0.000292 | 0.00092 | 16384 | 0.28046 | 272.55289 | 6.34181 | 242.07123 | 84.54093 |
| 15 | 0.000255 | 0.0008 | 32768 | 0.28046 | 272.55289 | 6.34181 | 242.07123 | 84.54093 |
| 16 | 0.000232 | 0.00066 | 65536 | 0.28046 | 272.55289 | 6.34181 | 242.07123 | 84.54093 |
| 17 | 0.000326 | 0.00086 | 131072 | NA | NA | NA | NA | NA |
| 18 | 0.000306 | 0.00071 | 262144 | NA | NA | NA | NA | NA |
| 19 | 0.000291 | 0.0006 | 524288 | NA | NA | NA | NA | NA |
| 20 | 0.000275 | 0.0005 | 1048576 | NA | NA | NA | NA | NA |
| 21 | 0.000268 | 0.00043 | 2097152 | NA | NA | NA | NA | NA |

NA: not applicable.

Table II shows the physical properties of the gas mixtures tested (Mixture A: air, i.e. N₂/O₂; Mixture B: He/O₂) used in the preceding simulation.

TABLE II

| Gas concentration | Viscosity | Density | Kinematic viscosity |
|---|---|---|---|
| (Vol %) | (kg/m · s) | (kg/m³) | (m²/s) |
| Mixture A (air) | | | |
| N₂/O₂ = 78/22 | 1.809 × 10⁻⁵ | 1.201 | 1.506 × 10⁻⁵ |
| Mixture B | | | |
| He/O₂ = 78/22 | 2.152 × 10⁻⁵ | 0.422 | 5.100 × 10⁻⁵ |

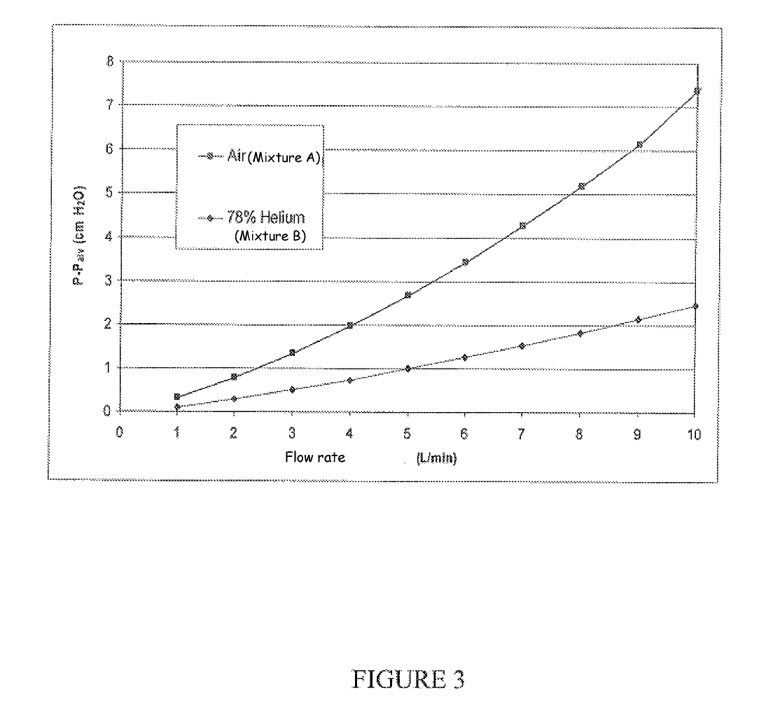

FIG. 3 shows the change in the pressure drop between the mouth and the alveolar zone of an individual for various flow rates (L/min) of the mixtures A (i.e. air) and B (i.e. helium/O₂) tested.

It is observed that the loss of energy is greater in the case of the inhalation of air (mixture A) than in the case of the inhalation of a helium-oxygen mixture (mixture B).

In the case of a mixture composed of helium, oxygen and several ppm of NO, typically less than 1000 ppm by volume of NO, according to the invention, the physical properties will not be significantly modified.

The respiratory effort provided by the patient will therefore be lower in the case of an NO/He/O₂ mixture according to the invention, which will then enable the patient to inhale a greater volume of gas for the same inspiratory effort provided by the patient.

The nitric oxide (NO) can therefore be inhaled more deeply by the patient and can therefore act more successfully in combating the bacteria.

This therefore confirms the advantage of using an NO/He/O₂ mixture according to the invention instead of an NO/N₂/O₂ (i.e. air/NO) mixture according to the prior art since such an NO/He/O₂ mixture offers a lower gas flow resistance in the respiratory airways of the patient, reducing the respiratory work and thus providing, at the alveolar level, a higher concentration of NO which conserves the bactericidal efficacy thereof.

In other words, it is particular advantageous to use an NO/He gas composition according to the invention for manufacturing an inhalable medicament intended to treat a bacterial infection affecting all or some of the respiratory airways of an individual.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1.-10. (canceled)

11. A method of reducing the risk of or treating at least one bacterial infection affecting all or some of a respiratory airway of a patient comprising the step of administering a gas composition containing nitric oxide (NO) and helium (He) to the respiratory airway of the patient.

12. The method of claim 11, wherein the respiratory airway comprises a bronchial tree and a lung.

13. The method of claim 11, wherein the gas composition comprises a content of NO between 10 and 5000 ppm by volume.

14. The method of claim 13, wherein the content of NO is between 100 and 1000 ppm by volume.

15. The method of claim 11, wherein the gas composition additionally contains at least 21% by volume of oxygen.

16. The method of claim 11, wherein the gas composition additionally contains nitrogen (N₂).

17. The method of claim 11, wherein the patient is a human being.

18. The method of claim 11, further comprising a step wherein the gas composition is diluted with an oxygen-containing gas in a ventilation circuit of a respirator or of a medical ventilator.

19. The method of claim 11, wherein the gas composition is packaged in a gas cylinder.

20. The method of claim 11, wherein the gas composition consists of helium, nitrogen and NO.
